(19) United States
(12) Patent Application Publication
(10) Pub. No.: US 2007/0105828 A1
(43) Pub. Date: May 10, 2007

(54) Novel polymorph form M of mifepristone and process for its preparation

(75) Inventors: Narendra Shriram Joshi, Navi Mumbai (IN); Anil Shahaji Khile, New Panvel (IN); Nitin Sharad Chandra Pradhan, Thane (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/506,223

(22) Filed: Aug. 18, 2006

Related U.S. Application Data
(60) Provisional application No. 60/713,019, filed on Aug. 31, 2005.

(30) Foreign Application Priority Data
Aug. 19, 2005 (IN) 978/MUM/2005

(51) Int. Cl. A61K 31/56; C07J 7/00

(52) U.S. Cl. 514/177; 552/592

(57) ABSTRACT

Mifepristone substantially in polymorph form M is provided. Also provided is a process for the preparation of polymorph form M of mifepristone comprising the steps of (a) dissolving crude mifepristone in a polar solvent at an elevated temperature to obtain a clear solution; (b) cooling the solution to a temperature and for a time period sufficient to form a precipitate of mifepristone crystals; and (c) isolating the precipitate of mifepristone crystals to obtain the polymorph form M of mifepristone.

### PRIORITY

This application claims the benefit under 35 U.S.C. §119 to U.S. Provisional Application No. 60/713,019, filed on Aug. 31, 2005, and entitled “NOVEL POLYMORPHIC FORM M OF MIFEPRISTONE AND PROCESS FOR ITS PREPARATION” and to Indian Provisional Application No. 978/MUM/2005, filed on Aug. 19, 2005, and entitled “NOVEL POLYMORPHIC FORM M OF MIFEPRISTONE AND PROCESS FOR ITS PREPARATION”, the contents of each of which are incorporated by reference herein.

### BACKGROUND OF THE INVENTION

1. Technical Field

The present invention generally relates to a novel polymorph of mifepristone and to a process for its preparation.

2. Description of the Related Art

Mifepristone, also known as (11β,17β)-11-[4-(N,N-dimethylamino)phenyl]-17-hydroxy-17-(1-propynyl)estra-4,9-diene-3-one, can be represented by the structure of Formula I. 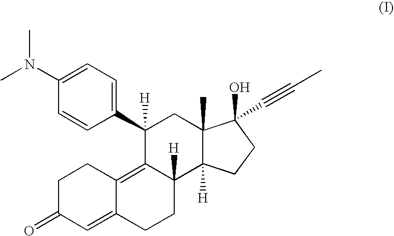
Generally, mifepristone is a potent 19-norsteroid that blocks the action of the female hormone progesterone of Formula II, which is necessary for initiating and sustaining pregnancy of a female.

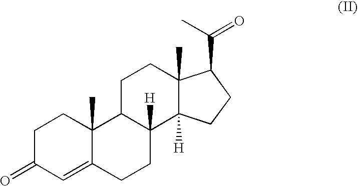

In the absence of the progesterone of Formula II, the uterine lining breaks down and bleeding occurs, thus resulting in the termination of the pregnancy. Mifepristone is sold under the brand name Mifegyne® in the United Kingdom and Mifeprex® in the United States. See, e.g., The Merck Index, Thirteenth Edition, 2001, p. 1103, monograph 6209.

Mifepristone, when used with a small amount of a synthetic prostaglandin (misoprostol), can terminate early pregnancies (up to 6 to 8 weeks) effectively and safely (see, e.g., J. Indian Inst. Sci., May-June 2001, vol. 81, pp. 287-298). Thus, mifepristone can be an alternative to surgical termination of pregnancy. In addition to termination of early pregnancy, mifepristone is useful for preparing women for surgical abortion as it promotes dilation of the uterine cervix as effectively as the prostaglandins, but with fewer side effects. It has also been used to induce labor, after spontaneous death of the fetus in the uterus. Mifepristone has an antiglucocorticoid action with an affinity for the glucocorticoid receptor three times greater than that of dexamethasone. Hence mifepristone may also be used to treat patients with overreactive adrenal glands, known as Cushing's syndrome, which may arise from inoperable tumors.

U.S. Pat. No. 4,386,085 (the '085 patent) discloses mifepristone starting from estra-5(10), 9(11)-diene-3,17-dione 3-ethylene acetal. The '085 patent discloses the purification of mifepristone by column chromatography using cyclohexane-ethyl acetate (7:3) mixture as an eluent. However, a drawback to the use of column chromatography is its unsuitability for industrial use.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predicable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. Additionally, polymorphic forms of the same drug substance or active pharmaceutical ingredient, can be administered by itself or formulated as a drug product (also known as the final or finished dosage form), and are well known in the pharmaceutical art to affect, for example, the solubility, stability, flowability, tractability and compressibility of drug substances and the safety and efficacy of drug products.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic. A new polymorphic form of mifepristone has now been discovered.

### SUMMARY OF THE INVENTION

In accordance with one embodiment of the present invention, a process for the preparation of a polymorph form M of mifepristone is provided comprising the steps of:

(a) dissolving crude mifepristone in a polar solvent at an elevated temperature to obtain a clear solution;

(b) cooling the solution to a temperature and for a time period sufficient to form a precipitate of mifepristone crystals; and

(c) isolating the mifepristone crystals to obtain the polymorph form M of mifepristone.

In accordance with a second embodiment of the present invention, mifepristone substantially in polymorph M form is provided.

In accordance with a third embodiment of the present invention, mifepristone substantially in polymorph M form is provided having an X-ray diffraction (XRD) pattern substantially in accordance with FIG. 1.

In accordance with a fourth embodiment of the present invention, mifepristone substantially in polymorph M form is provided and exhibiting characteristic peaks (expressed in degrees 2θ±0.2°θ) at approximately one or more of the positions: about 17.26 and about 18.50.

In accordance with a fifth embodiment of the present invention, mifepristone substantially in polymorph M form is provided having an Infrared (IR) spectrum substantially in accordance with FIG. 2.

In accordance with a sixth embodiment of the present invention, mifepristone substantially in polymorph M form is provided having a differential scanning calorimetry (DSC) thermogram substantially in accordance with FIG. 3.

In accordance with a seventh embodiment, substantially pure mifepristone substantially in polymorph form M is provided.

In accordance with an eighth embodiment, a pharmaceutical composition is provided comprising a therapeutically effective amount of mifepristone substantially in polymorph form M and one or more pharmaceutically acceptable carriers.

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in tablet granulations. Such compounds include, by way of example and without limitation, acacia alginic acid, tragacanth, carboxymethylcellulose sodium, poly(vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

The term “diluent” or “filler” as used herein is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, polyvinylpyrrolidone (PVP), tyloxapol (a nonionic liquid polymer of the alkyl aryl polyether alcohol type, also known as superinone or triton), combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### BRIEF DESCRIPTION THE DRAWINGS

FIG. 1 is a characteristic powder XRD pattern of polymorph form M of mifepristone.

FIG. 2 is a characteristic IR spectrum of polymorph form M of mifepristone.

FIG. 3 is a characteristic DSC thermogram of polymorph form M of mifepristone.

### DESCRIPTION OF THE PREFERRED EMBODIMENTS

The present invention is directed to a novel polymorph form of mifepristone, designated polymorph form M. The polymorph form M of mifepristone has at least one, and preferably all, of the following properties:

(a) a melting point in the range of about 191° C. to about 196° C.;

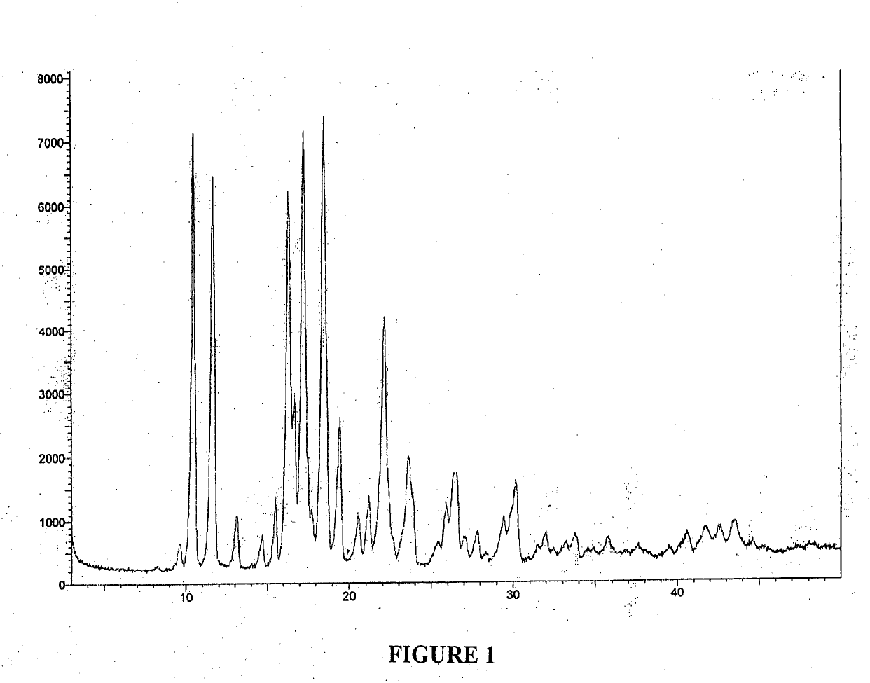

(b) a XRD pattern substantially in accordance with FIG. 1;

(c) a XRD pattern comprising characteristic peaks (expressed in degrees 2θ±0.2°θ) summarized in Table I below:

TABLE I

| Sr. No. | Angle (2θ) | Relative Intensity (%) |
|---|---|---|
| 1 | 9.62 | 6.10 |
| 2 | 10.32 | 42.52 |
| 3 | 10.48 | 95.28 |
| 4 | 11.67 | 87.00 |
| 5 | 13.09 | 11.33 |
| 6 | 14.68 | 6.93 |
| 7 | 15.52 | 13.22 |
| 8 | 16.32 | 81.67 |
| 9 | 16.40 | 76.34 |
| 10 | 16.68 | 36.92 |
| 11 | 17.26 | 95.99 |
| 12 | 17.52 | 21.04 |
| 13 | 17.72 | 12.03 |
| 14 | 18.40 | 79.36 |
| 15 | 18.50 | 100.00 |
| 16 | 19.42 | 32.13 |
| 17 | 20.52 | 10.74 |
| 18 | 20.59 | 9.42 |
| 19 | 21.14 | 15.36 |
| 20 | 22.1 | 55.02 |
| 21 | 22.40 | 14.77 |
| 22 | 23.56 | 23.14 |
| 23 | 23.65 | 22.97 |
| 24 | 23.86 | 15.33 |
| 25 | 23.93 | 12.14 |
| 26 | 25.91 | 13.69 |
| 27 | 26.29 | 17.28 |
| 28 | 26.48 | 23.51 |
| 29 | 26.62 | 15.94 |
| 30 | 27.83 | 6.81 |
| 31 | 29.41 | 9.17 |
| 32 | 29.80 | 10.36 |
| 33 | 30.13 | 17.90 |
| 34 | 30.20 | 16.95 |
| 35 | 43.39 | 7.14 |
| 36 | 43.52 | 7.49 |

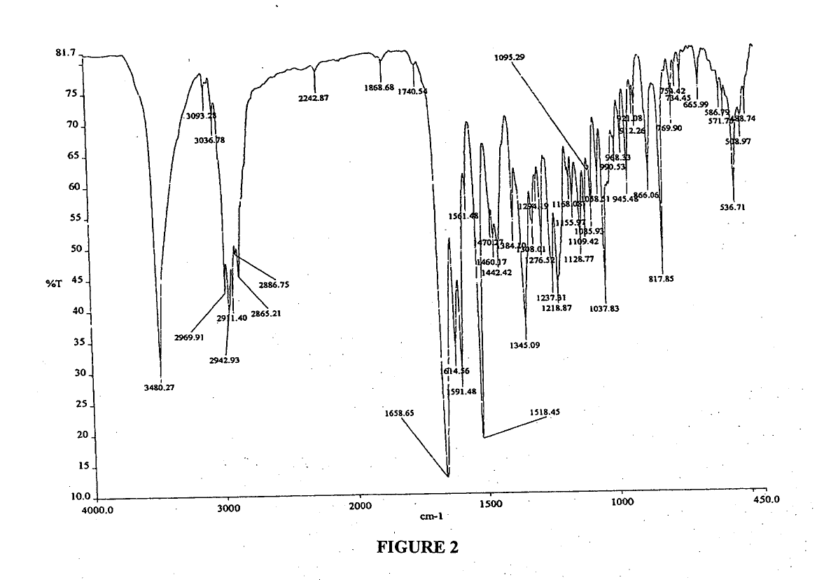

(d) an IR spectrum substantially in accordance with FIG. 2;

(e) an IR absorption spectrum having absorption bands in the region of about 3480, 3093, 3036, 2969, 2942, 2911, 2886, 2865, 2242, 1868, 1658, 1614, 1591, 1518, 1460, 1442, 1345, 1276, 1237, 1218, 1128, 1037, 945, 866, 817, 769, 666 and 536±2 cm⁻¹; and/or

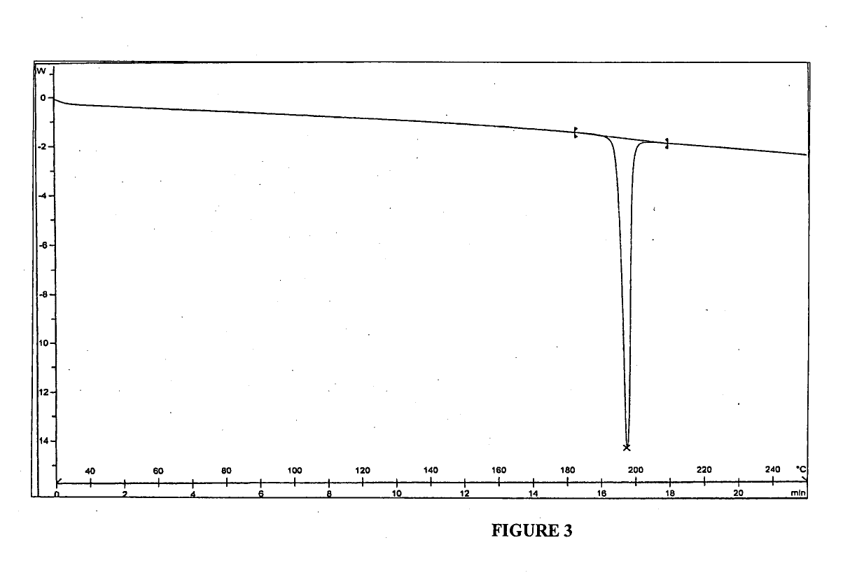

(f) a DSC thermogram substantially in accordance with FIG. 3.

Generally, the polymorph form M of mifepristone can be obtained by at least (a) dissolving crude mifepristone in a polar solvent at an elevated temperature to obtain a clear solution; (b) cooling the solution to a temperature and for a time period sufficient to form a precipitate of mifepristone crystals; and (c) isolating the mifepristone crystals to obtain the polymorph form M of mifepristone.

Mifepristone and processes for its preparation are known in the art. See, e.g., U.S. Pat. No. 4,386,085, the contents of which are incorporated by reference herein. In one embodiment, the crude mifepristone is obtained by hydrolysis of (5α,11β,17β)-11-[4-(Dimethylamino)phenyl]-5,17-dihydroxy-17-(1-propynyl)estra-9(11)-en-3-one ethylene acetal.

Suitable polar solvents for use in step (a) of the process of the present invention include C₁-C₁₈ alcohols and preferably C₁-C₆ alcohols and the like and mixtures thereof. Representative examples of alcohols include methanol, ethanol, isopropyl alcohol, n-propyl alcohol and the like and mixtures thereof. The temperature for dissolution will ordinarily range from about 60° C. to about 90° C., preferably from about 70° C. to about 85° C., and most preferably from about 75° C. to about 80° C.

In step (b), the solution is cooled is to a temperature and for a time period sufficient to form a precipitate of mifepristone crystals. Generally, the solution may be cooled gradually to about room temperature. The time period required to cool to room temperature can range from about 1 hour to about 3 hours and most preferably about 1.5 hours to about 2 hours. The cooled solution can be stirred at room temperature for about 4 hours to about 7 hours and most preferably about 5 hours to about 6 hours.

Next, the solution can be further cooled to about 0° C. to about −20° C., preferably from about 0° C. to about −15° C., and most preferably from about −5° C. to about −10° C. to form a precipitate of mifepristone crystals. The time period to obtain the precipitate can range from about 9 hours to about 13 hours and most preferably about 10 hours to about 11 hours.

The precipitate of mifepristone crystals can then be isolated by conventional techniques, e.g., filtration, and dried to obtain the novel polymorph form M of mifepristone. In one embodiment, the precipitate of mifepristone crystals can be isolated by filtration employing a filtration media of, for example, a silica gel or celite. The product can be filtered at a temperature ordinarily ranging from about 0° C. to about −20° C., preferably from about 0° C. to about −15° C., and most preferably from about −5° C. to about −10° C. The isolated product can be dried at a temperature ranging from about 40° C. to about −80° C., preferably from about 50° C. to about 55° C.

Yet another aspect of the present invention is directed to pharmaceutical compositions containing at least the novel polymorph form M of mifepristone disclosed herein. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like. The novel polymorph form M of mifepristone disclosed herein also may be administered as suppositories, ophthalmic ointments and suspensions, and parenteral suspensions, which are administered by other routes. The dosage forms may contain the novel polymorph form M of mifepristone disclosed herein as is or, alternatively, may contain the novel polymorph form M of mifepristone disclosed herein as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain the novel polymorph form M of mifepristone disclosed herein within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants such as magnesium and calcium stearate and sodium stearyl fumarate; flavorings; sweeteners; preservatives; pharmaceutically acceptable dyes and glidants such as silicon dioxide.

In one embodiment, the novel polymorph form M of mifepristone disclosed herein for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. The particle sizes of the novel polymorph form M of mifepristone can be obtained by, for example, any milling, grinding, micronizing or other particle size reduction method known in the art to bring the solid state polymorph form M of mifepristone into any of the foregoing desired particle size range.

Actual dosage levels of the polymorph form M of mifepristone of the invention may be varied to obtain an amount of mifepristone that is effective to obtain a desired therapeutic response for a particular composition and method of administration for treatment of a mammal. The selected dosage level therefore depends upon such factors as, for example, the desired therapeutic effect, the route of administration, the desired duration of treatment, and other factors. The total daily dose of the polymorph form M of mifepristone of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc.

The following examples are provided to enable one skilled in the art to practice the invention and are merely illustrative of the invention. The examples should not be read as limiting the scope of the invention as defined in the claims.

### EXAMPLE 1

120.0 g of crude mifepristone obtained from hydrolysis of 100.0 g of (5α,11β,17β)-11-[4-(Dimethylamino)phenyl]-5,17-dihydroxy-17-(1-propynyl)estra-9(11)-en-3-one ethylene acetal [prepared from 17β-17-hydroxy-17-(1-propynyl)estra-5(10),9(11)-dien-3-one ethylene acetal by epoxidation followed by reaction with Grignard complex of 4-bromo-N,N-dimethyl aniline (or according to Example 4 of U.S. Pat. No. 4,386,085) was dissolved in 300 ml isopropyl alcohol and the solution was heated to 75-80° C. The solution was then cooled gradually to room temperature (2 hours) and stirred at room temperature overnight. The solution was further cooled to −5 to −10° C. and maintained for 1 hour. The solid obtained was collected by filtration followed by washing with chilled isopropyl alcohol and n-hexane. The product was dried under vacuum at 50-55° C.

Yield: 59.0 gm

HPLC Purity: 99.29%

The compound showed a sharp melting point at 192.3° C.-193.4° C. The XRD pattern and IR absorption spectrum of the final compound are set forth in FIGS. 1 and 2 and was recorded and identified as polymorphic form M.

### EXAMPLE 2

112.0 g of crude mifepristone obtained from hydrolysis of 100.0 g of (5α,11β,17β)-11-[4-(Dimethylamino)phenyl]-5,17-dihydroxy-17-(1-propynyl)estra-9(11)-en-3-one ethylene acetal [prepared from 17β-17-hydroxy-17-(1-propynyl)estra-5(10),9(11)-dien-3-one ethylene acetal by epoxidation followed by reaction with Grignard complex of 4-bromo-N,N-dimethyl aniline (or according to Example 4 of U.S. Pat. No. 4,386,085) was dissolved in 300 ml isopropyl alcohol heated and the solution was heated to 75-80° C. The solution was then cooled gradually to room temperature (2 hrs) and stirred at room temperature for 5 hours. The solution was further cooled to −5 to −10° C. and maintained for 10 hours. The solid obtained was collected by filtration followed by washing with chilled isopropyl alcohol and n-hexane. The product was dried under vacuum at 50-55° C.

Yield: 62.0 g

HPLC Purity: 99.41%

The compound showed a sharp melting point at 192.2° C.-193.4° C. The XRD pattern and IR absorption spectrum of the final compound are set forth in FIGS. 1 and 2 and was recorded and identified as polymorphic form M.

While the above description contains many specifics, these specifics should not be construed as limitations of the invention, but merely as exemplifications of preferred embodiments thereof. Those skilled in the art will envision many other embodiments within the scope and spirit of the invention as defined by the features and advantages appended hereto.

What is claimed is:

1. Mifepristone substantially in polymorph form M.

2. The mifepristone substantially in polymorph form M of claim 1, further characterized by a X-ray diffraction pattern (XRD) substantially in accordance with FIG. 1.

3. The mifepristone substantially in polymorph form M of claim 1, further characterized by characteristic peaks (expressed in degrees 2θ±0.2°θ) at approximately one or more of the positions: about 17.26 and about 18.50.

4. The mifepristone substantially in polymorph form M of claim 1, further characterized by having an Infrared (IR) spectrum substantially in accordance with FIG. 2.

5. The mifepristone substantially in polymorph form M of claim 1, further characterized by a differential scanning calorimetry (DSC) thermogram substantially in accordance with FIG. 3.

6. Substantially pure polymorph form M of mifepristone.

7. A process for the preparation of polymorph form M of mifepristone, the process comprising the steps of:
(a) dissolving crude mifepristone in a polar solvent at an elevated temperature to obtain a clear solution;
(b) cooling the solution to a temperature and for a time period sufficient to form a precipitate of mifepristone crystals; and
(c) isolating the precipitate of mifepristone crystals to obtain the polymorph form M of mifepristone.

8. The process of claim 7, wherein the polar solvent in step (a) comprises an alcohol.

9. The process of claim 7, wherein the polar solvent in step (a) comprises a C₁-C₁₈ alcohol.

10. The process of claim 7, wherein the polar solvent in step (a) comprises an alcohol selected from the group consisting of methyl alcohol, ethyl alcohol, isopropyl alcohol, propyl alcohol and mixtures thereof.

11. The process of claim 7, wherein the amount of polar solvent in step (a) is about 2.5 to about 2.7 volume of the crude mifepristone.

12. The process of claim 7, wherein the temperature in step (a) is about 75 to about 80° C.

13. The process of claim 7, wherein the step of isolating comprises filtering the precipitate of mifepristone crystals.

14. The process of claim 13, wherein the step filtering is carried out with a filter aid.

15. The process of claim 14, wherein the filter aid is celite.

16. The process of claim 7, wherein the temperature of cooling the solution is about −5 to about −10° C.

17. The process of claim 7, further comprising drying the isolated polymorph form M of mifepristone.

18. The process of claim 7, further comprising drying the isolated polymorph form M of mifepristone at a temperature of about 50° C. to about 55° C.

19. A pharmaceutical composition comprising the polymorph form M of mifepristone of claim 1 and one or more pharmaceutically acceptable excipients.

20. A pharmaceutical composition comprising the polymorph form M of mifepristone of claim 3 and one or more pharmaceutically acceptable excipients.
